(19) United States
(12) Patent Application Publication
(10) Pub. No.: US 2009/0076397 A1
(43) Pub. Date: Mar. 19, 2009

(54) Adherent Emergency Patient Monitor

(75) Inventors: Imad Libbus, Saint Paul, MN (US); Badri Amurthur, Los Gatos, CA (US); Mark J. Bly, Falcon Heights, MN (US); Kristofer J. James, Eagan, MN (US); Yatheendhar D. Manicka, Woodbury, MN (US); Scott T. Mazar, Woodbury, MN (US); Jerry S. Wang, Blaine, MN (US)

Correspondence Address: TOWNSEND AND TOWNSEND AND CREW, LLP, TWO EMBARCADERO CENTER, EIGHTH FLOOR, SAN FRANCISCO, CA, 94111-3834, US

(73) Assignee: Corventis, Inc., San Jose, CA (US)

(21) Appl. No.: 12/209,259

(22) Filed: Sep. 12, 2008

Related U.S. Application Data
(60) Provisional application No. 60/972,537, filed on Sep. 14, 2007.
(60) Provisional application No. 60/972,581, filed on Sep. 14, 2007.
(60) Provisional application No. 61/055,666, filed on May 23, 2008.

(51) Int. Cl. A61B 5/02; A61B 5/0402; A61B 5/08

(52) U.S. Cl. 600/484; 600/509

(57) ABSTRACT

An adherent device comprises an adhesive patch to adhere to a skin of the patient. At least two electrodes are connected to the patch and capable of electrically coupling to the patient. Electrocardiogram circuitry can be coupled to at the least two electrodes to measure an electrocardiogram signal of the patient. An accelerometer can be mechanically coupled to the adhesive patch to generate an accelerometer signal in response to at least one of an activity or a position of the patient. A processor comprising a tangible medium can be configured to communicate with the electrocardiogram circuitry and the accelerometer to generate an alarm signal in response to the electrocardiogram signal and the accelerometer signal.

### CROSS-REFERENCES TO RELATED APPLICATIONS

The present application claims the benefit under 35 USC 119(e) of U.S. Provisional Application Nos. 60/972,581 and 60/972,537 both filed Sep. 14, 2007 and 61/055,666 filed May 23, 2008; the full disclosures of which are incorporated herein by reference in their entirety.

The subject matter of the present application is related to the following applications: 60/972,512; 60/972,329; 60/972,354; 60/972,616; 60/972,363; 60/972,343; 60/972,629; 60/972,316; 60/972,333; 60/972,359; 60/972,336; 60/972,340 all of which were filed on Sep. 14, 2007; 61/046,196 filed Apr. 18, 2008; 61/047,875 filed Apr. 25, 2008; 61/055,645, 61/055,656, 61/055,662 all filed May 23, 2008; and 61/079,746 filed Jul. 10, 2008.

The following applications are being filed concurrently with the present application, on Sep. 12, 2008: Attorney Docket Nos. 026843-000110US entitled “Multi-Sensor Patient Monitor to Detect Impending Cardiac Decompensation Prediction”; 026843-000220US entitled “Adherent Device with Multiple Physiological Sensors”; 026843-000410US entitled “Injectable Device for Physiological Monitoring”; 026843-000510US entitled “Delivery System for Injectable Physiological Monitoring System”; 026843-000620US entitled “Adherent Device for Cardiac Rhythm Management”; 026843-000710US entitled “Adherent Device for Respiratory Monitoring”; 026843-000810US entitled “Adherent Athletic Monitor”; 026843-001320US entitled “Adherent Device with Physiological Sensors”; 026843-001410US entitled “Medical Device Automatic Start-up upon Contact to Patient Tissue”; 026843-001900US entitled “System and Methods for Wireless Body Fluid Monitoring”; 026843-002010US entitled “Adherent Cardiac Monitor with Advanced Sensing Capabilities”; 026843-002410US entitled “Adherent Device for Sleep Disordered Breathing”; 026843-002710US entitled “Dynamic Pairing of Patients to Data Collection Gateways”; 026843-003010US entitled “Adherent Multi-Sensor Device with Implantable Device Communications Capabilities”; 026843-003110US entitled “Data Collection in a Multi-Sensor Patient Monitor”; 026843-003210US entitled “Adherent Multi-Sensor Device with Empathic Monitoring”; 026843-003310US entitled “Energy Management for Adherent Patient Monitor”; and 026843-003410US entitled “Tracking and Security for Adherent Patient Monitor.”

### BACKGROUND OF THE INVENTION

1. Field of the Invention

The present invention relates to patient monitoring. Although embodiments make specific reference to monitoring electrocardiogram signals with an adherent patch, the system methods and device described herein may be applicable to many applications in which physiological monitoring is used, for example wireless physiological monitoring for extended periods.

Patients are often treated for diseases and/or conditions associated with a compromised status of the patient, for example a compromised physiologic status. The compromised status of the patient can result from age and/or disease. In some instances, a patient may report symptoms that require diagnosis to determine the underlying cause or the patient may be at risk for an adverse event, such that monitoring is indicated. For example, a patient may report fainting or dizziness that requires diagnosis, in which long term monitoring of the patient can provide useful information as to the physiologic status of the patient. In some instances, a patient may have suffered a heart attack and require care and/or monitoring after release from the hospital. One example of a device to provide long term monitoring of a patient is the Holter monitor, or ambulatory electrocardiography device.

Work in relation to embodiments of the present invention suggests that known methods and apparatus for long term monitoring of patients may be less than ideal. At least some of the known devices may not collect the right kinds of data to treat patients optimally. Additionally, patients who are at risk, may not receive emergency and/or additional care in a timely manner such that the patient's health may be compromised. In at least some instances, devices that are worn by the patient may be somewhat uncomfortable, which may lead to patients not wearing the devices and not complying with direction from the health care provider, such that data collected may be less than ideal. Although implantable devices may be used in some instances, many of these devices can be invasive and/or costly, and may suffer at least some of the shortcomings of known wearable devices.

Work in relation to embodiments of the present invention also suggests that emergency patient monitors may be less than ideal. Patient's who are treated by a first responder can be connected to sensor that measure heart rate and other signals, yet some sensors may interfere with access to the patient in emergency situations. Additionally, some sensors may connect to multiple locations of the patient and may require time to connect to the patient, thereby adding to the time and complexity of patient treatment in some situations where time may be critical.

Therefore, a need exists for improved patient monitoring. Ideally, such improved patient monitoring would avoid at least some of the short-comings of the present methods and devices.

2. Description of the Background Art

The following U.S. patents and Publications may describe relevant background art: U.S. Pat. Nos. 4,121,573; 4,955,381; 4,981,139; 5,080,099; 5,353,793; 5,511,553; 5,544,661; 5,558,638; 5,724,025; 5,772,586; 5,862,802; 6,047,203; 6,117,077; 6,129,744; 6,225,901; 6,385,473; 6,416,471; 6,454,707; 6,454,708; 6,527,711; 6,527,729; 6,551,252; 6,595,927; 6,595,929; 6,605,038; 6,645,153; 6,821,249; 6,980,851; 7,020,508; 7,054,679; 7,153,262; 2003/0092975; 2003/0149349; 2005/0113703; 2005/0131288; 2006/0010090; 2006/0031102; 2006/0089679; 2006/122474; 2006/0155183; 2006/0224051; 2006/0264730; 2006/0264767; 2006/0276714; 2007/0167848; 2007/0021678; 2006/0030781; 2006/0030782; and 2007/0038038.

### BRIEF SUMMARY OF THE INVENTION

The present invention relates to patient monitoring. Although embodiments make specific reference to monitoring impedance and electrocardiogram signals with an adherent patch, the system methods and device described herein may be applicable to any application in which physiological monitoring is used, for example wireless physiological monitoring for extended periods. In many embodiments, the use of multiple sensors on an adherent patch can decrease false positives and decrease false negatives while increasing both sensitivity and specificity of patient diagnosis. In addition, several sensors can be connected to the patient with the adherent patch quickly, so as to allow a first responder to care to the patient more rapidly. In many embodiments, the electronic components can share resources, for example a processor and/or batteries, so as to decrease the footprint, or size of the device. This decrease in size of the device can provide improved patient comfort and/or access to patient, for example in critical care situations. In many embodiments, the adherent device comprises a processor configured to communicate with electrocardiogram circuitry and an accelerometer to generate an alarm signal in response to the electrocardiogram signal and the accelerometer signal center, such that the patient can receive appropriate care.

In a first aspect, embodiments of the present invention provide an adherent device to monitor a person, for example a person who may be at risk such as a soldier, minor, fire fighter, elderly person and/or person with diminished health such as a patient. The device comprises an adhesive patch to adhere to a skin of the person. At least two electrodes are connected to the patch and capable of electrically coupling to the person. Electrocardiogram circuitry can be coupled to at the least two electrodes to measure an electrocardiogram signal of the person. An accelerometer can be mechanically coupled to the adhesive patch to generate an accelerometer signal in response to at least one of an activity or a position of the person. A processor comprising a tangible medium can be configured to communicate with the electrocardiogram circuitry and the accelerometer to generate an alarm signal in response to the electrocardiogram signal and the accelerometer signal.

In many embodiments, the processor is configured to transmit at least one of the electrocardiogram signal or the accelerometer signal in real time to the remote center and/or a remote care giver in response to the alarm. The processor can be configured to generate the alarm signal in response to at least one of a cardiac rhythm disorder, a fall or a respiratory distress of the person.

In many embodiments, the processor can be configured to generate the alarm signal in response to a detected person fall from the accelerometer signal The processor can be configured to generate the alarm signal in response to an increased heart rate from the electrocardiogram signal and a decreased person activity from the accelerometer signal.

In many embodiments, the adherent device comprises respiration circuitry to measure a respiration signal of the person, and the processor is configured to generate the alarm signal in response to a respiratory distress from the respiration signal. The processor can be configured to combine the electrocardiogram signal, the accelerometer signal and respiration signal to generate the alarm signal. In some embodiments, the processor is configured to generate the alarm signal in response to an abnormal respiratory rate from the respiration signal and a decreased person activity from the accelerometer signal. In some embodiments, the processor is configured to generate the alarm signal in response to an abnormal heart rate from the electrocardiogram signal, an abnormal respiratory rate from the respiration signal, and a decreased activity measured from the accelerometer signal. In specific embodiments, the respiration circuitry comprises at least one of an impedance circuitry or a strain gauge.

In many embodiments, combining comprises using the at least two of the electrocardiogram signal, the accelerometer signal, or the respiration signal to look up a value in a previously existing array. In some embodiments, combining comprises at least one of adding, subtracting, multiplying, scaling or dividing the at least two of the electrocardiogram signal, the accelerometer signal, or the respiration signal. In specific embodiments, at least two of the electrocardiogram signal, the accelerometer signal, or the respiration signal are combined with at least one of a weighted combination, a tiered combination or a logic gated combination, a time weighted combination or a rate of change.

In many embodiments, the adhesive patch is mechanically coupled to the at least two electrodes, the electrocardiogram circuitry, the accelerometer, the respiration circuitry and the processor, such that the patch is capable of supporting the at least two electrodes, the electrocardiogram circuitry, the respiration circuitry, the accelerometer and the processor when the adherent patch is adhered to the skin of the person.

In many embodiments, the adherent device comprises a wireless communication circuitry coupled to the processor transmit at least one of the electrocardiogram signal, the respiration signal, or the accelerometer signal to a remote center with a communication protocol. The wireless communication circuitry can be configured to transmit the at least one of the electrocardiogram signal, the respiration signal or the accelerometer signal to the remote center with a single wireless hop from the wireless communication circuitry to an intermediate device and a wireless hop from the intermediate device to the remote center.

In another aspect, embodiments of the present invention provide a method of monitoring a frail person. An adhesive patch is adhered to a skin of the person, such that at least two electrodes connected to the patch are coupled to the skin of the person. An electrocardiogram signal of the person is measured with electrocardiogram circuitry coupled to at the least two electrodes. An accelerometer signal is measured in response to at least one of an activity or a position of the person with an accelerometer mechanically coupled to the adhesive patch. An alarm signal is generated in response to the electrocardiogram signal and the accelerometer signal with a processor comprising a tangible medium and in communication with the electrocardiogram circuitry and the accelerometer.

In many embodiments, at least one of the electrocardiogram signal or the accelerometer signal is transmitted in real time to the remote center and/or a remote care giver in response to the alarm. The alarm signal may be generated in response to at least one of a cardiac rhythm disorder, a fall or a respiratory distress of the person.

In many embodiments, the alarm signal is generated in response to a detected person fall from the accelerometer signal. The alarm signal can be generated in response to an increased heart rate measured with the electrocardiogram signal and a decreased person activity measured with the accelerometer signal.

In many embodiments, the alarm signal is generated in response to a decreased heart rate measured with the electrocardiogram signal and a decreased person activity measured with the accelerometer signal, so as to indicate at least one of a syncope and/or fainting of the person.

In many embodiments, a respiration signal of the person is measured with respiration circuitry, and the alarm signal is generated in response to a respiratory distress from the respiration signal. The electrocardiogram signal, the accelerometer signal and respiration signal can be combined to generate the alarm signal. The alarm signal can be generated in response to an abnormal respiratory rate from the respiration signal and a decreased person activity from the accelerometer signal. The alarm signal can be generated in response to an abnormal heart rate from the electrocardiogram signal, an abnormal respiratory rate from the respiration signal, and a decreased activity from the accelerometer signal. The adhesive patch can be mechanically coupled to the at two electrodes, the electrocardiogram circuitry, the accelerometer, the respiration circuitry and the processor, such that the patch supports the at least two electrodes, the electrocardiogram circuitry, the respiration circuitry, the accelerometer and the processor when the adherent patch is adhered to the skin of the person.

In many embodiments, at least one of the electrocardiogram signal, the respiration signal, or the accelerometer signal is transmitted wirelessly to a remote center with a communication protocol. The at least one of the electrocardiogram signal, the respiration signal or the accelerometer signal can be transmitted to the remote center with a single wireless hop from the wireless communication circuitry to an intermediate device and a wireless hop from the intermediate device to the remote center.

In another aspect, embodiments of the present invention provide an adherent device to monitor a person in an emergency situation. The device comprises an adhesive patch to adhere to a skin of the person. At least two electrodes are connected to the patch and capable of electrically coupling to the person. Electrocardiogram circuitry is coupled to at the least two electrodes to measure an electrocardiogram signal of the person. The device comprises temperature circuitry to measure a temperature of the patient. The device comprises respiration circuitry to measure a respiration signal of the person. A processor comprising a tangible medium is configured to communicate with the electrocardiogram circuitry and the respiration circuitry to generate an alarm signal in response to at least two of the electrocardiogram signal, the temperature signal and the respiration signal. Wireless communication circuitry can be coupled to the processor, the electrocardiogram circuitry and the accelerometer to transmit the alarm signal to a remote center with a communication protocol.

In many embodiments, the adherent device of claim comprises temperature circuitry to measure a temperature signal from the patient.

In many embodiments, the processor is configured to transmit at least one of the electrocardiogram signal or the respiration signal in real time to the remote center and/or a remote care giver in response to the alarm. The wireless communication circuitry can be configured to transmit at least one of the electrocardiogram signal or the respiration signal in real time to the remote center in response to the alarm signal. The respiration circuitry may comprise at least one of an impedance circuitry or a strain gauge.

In many embodiments, the processor is configured to generate the alarm signal in response to an abnormal heart rate from the electrocardiogram signal and an abnormal respiratory rate from the respiration signal.

In many embodiments, the adherent device comprise an accelerometer mechanically coupled to the adhesive patch to generate an accelerometer signal in response to at the least one of an activity or a position of the person.

In many embodiments, the processor is configured to combine at least two of the electrocardiogram signal, the accelerometer signal, the temperature signal and respiration signal to generate the alarm signal. The processor can be configured to combine the at least two of the e electrocardiogram signal, the accelerometer signal, the temperature signal or the respiration signal to look up a value in a previously existing array. The processor may be configured to combine with at least one of adding, subtracting, multiplying, scaling or dividing the at least two of the electrocardiogram signal, the accelerometer signal, or the respiration signal. The at least two of the electrocardiogram signal, the accelerometer signal, the temperature signal or the respiration signal can be combined with at least one of a weighted combination, a tiered combination or a logic gated combination, a time weighted combination or a rate of change.

In many embodiments, the adhesive patch is mechanically coupled to the at two electrodes, the electrocardiogram circuitry, the respiration circuitry, the accelerometer, the temperature circuitry and the processor, such that the patch is capable of supporting the at least two electrodes, the electrocardiogram circuitry, the respiration circuitry, the accelerometer, the temperature circuitry and the processor when the adherent patch is adhered to the skin of the person.

In another aspect, embodiments of the present invention provide a method of monitoring a person in an emergency situation. An adhesive patch is adhered to a skin of the person, such that at least two electrodes connected to the patch are coupled to the skin of the person. An electrocardiogram signal of the person is measured with electrocardiogram circuitry coupled to at the least two electrodes. A respiration signal of the person is measured with respiration circuitry. A temperature signal is measured from the person with temperature circuitry. An alarm signal is generated in response to the electrocardiogram signal and the respiration signal with a processor comprising a tangible medium in communication with the electrocardiogram circuitry and the respiration circuitry. The alarm signal is transmitted to a remote center with a communication protocol and wireless communication circuitry. At least one of the electrocardiogram signal or the respiration signal can be transmitted in real time to the remote center and/or a remote care giver in response to the alarm. The alarm signal can be generated with the processor in response to an abnormal heart rate from the electrocardiogram signal and an abnormal respiratory rate from the respiration signal.

In many embodiments, an accelerometer signal can be generated in response to at least one of an activity or a position of the person with an accelerometer mechanically coupled to the adhesive patch. The electrocardiogram signal, the accelerometer signal, the temperature signal and respiration signal can be combined to generate the alarm signal with the processor. The adhesive patch can be mechanically coupled to the at two electrodes, the electrocardiogram circuitry, the respiration circuitry, the accelerometer and the processor, such that the patch supports the at least two electrodes, the electrocardiogram circuitry, the respiration circuitry, the accelerometer and the processor when the adherent patch is adhered to the skin of the person.

### BRIEF DESCRIPTION OF THE DRAWINGS

FIG. 1A shows a patient and a monitoring system comprising an adherent device, according to embodiments of the present invention;

FIG. 1B shows a bottom view of the adherent device as in FIG. 1A comprising an adherent patch;

FIG. 1C shows a top view of the adherent patch, as in FIG. 1B;

FIG. 1D shows a printed circuit boards and electronic components over the adherent patch, as in FIG. 1C;

FIG. 1D1 shows an equivalent circuit that can be used to determine optimal frequencies for determining patient hydration, according to embodiments of the present invention;

FIG. 1E shows batteries positioned over the printed circuit board and electronic components as in FIG. 1D;

FIG. 1F shows a top view of an electronics housing and a breathable cover over the batteries, electronic components and printed circuit board as in FIG. 1E;

FIG. 1G shows a side view of the adherent device as in FIGS. 1A to 1F;

FIG. 1H shown a bottom isometric view of the adherent device as in FIGS. 1A to 1G;

FIGS. 1I and 1J show a side cross-sectional view and an exploded view, respectively, of the adherent device as in FIGS. 1A to 1H;

FIG. 1K shows at least one electrode configured to electrically couple to a skin of the patient through a breathable tape, according to embodiments of the present invention; and

FIG. 2A shows a method of monitoring a patient, according to embodiments of the present invention.

### DETAILED DESCRIPTION OF THE INVENTION

Embodiments of the present invention relate to patient monitoring. Although embodiments make specific reference to monitoring impedance and electrocardiogram signals with an adherent patch, the system methods and device described herein may be applicable to any application in which physiological monitoring is used, for example wireless physiological monitoring for extended periods.

In many embodiments, the adherent devices described herein may be used for 90 day monitoring, or more, and may comprise completely disposable components and/or reusable components, and can provide reliable data acquisition and transfer. In many embodiments, the patch is configured for patient comfort, such that the patch can be worn and/or tolerated by the patient for extended periods, for example 90 days or more. In many embodiments, the adherent patch comprises a tape, which comprises a material, preferably breathable, with an adhesive, such that trauma to the patient skin can be minimized while the patch is worn for the extended period. In many embodiments, the printed circuit board comprises a flex printed circuit board that can flex with the patient to provide improved patient comfort.

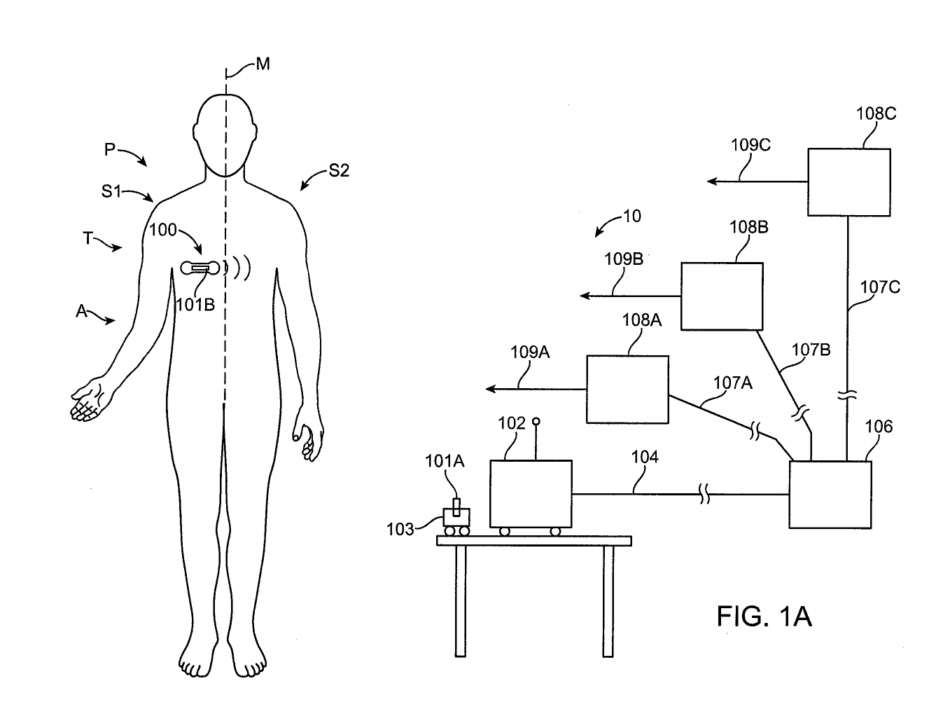

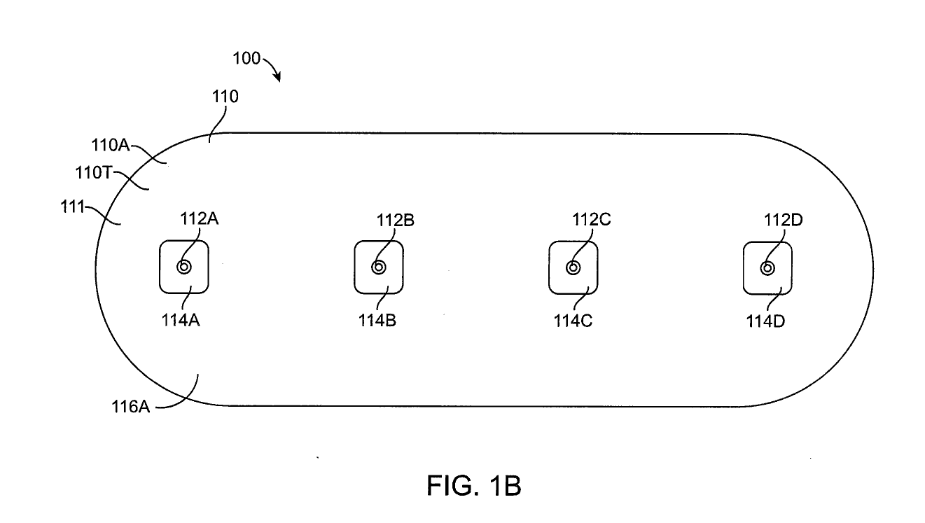

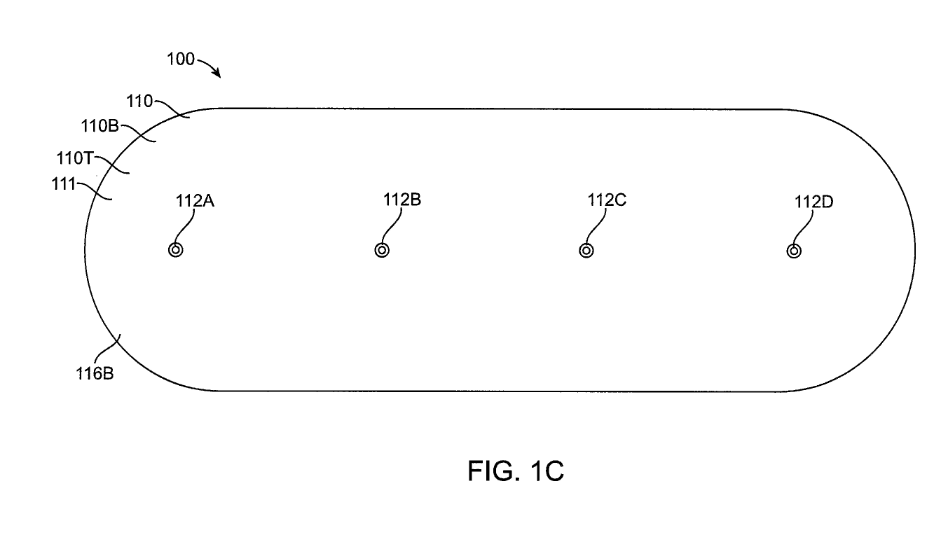

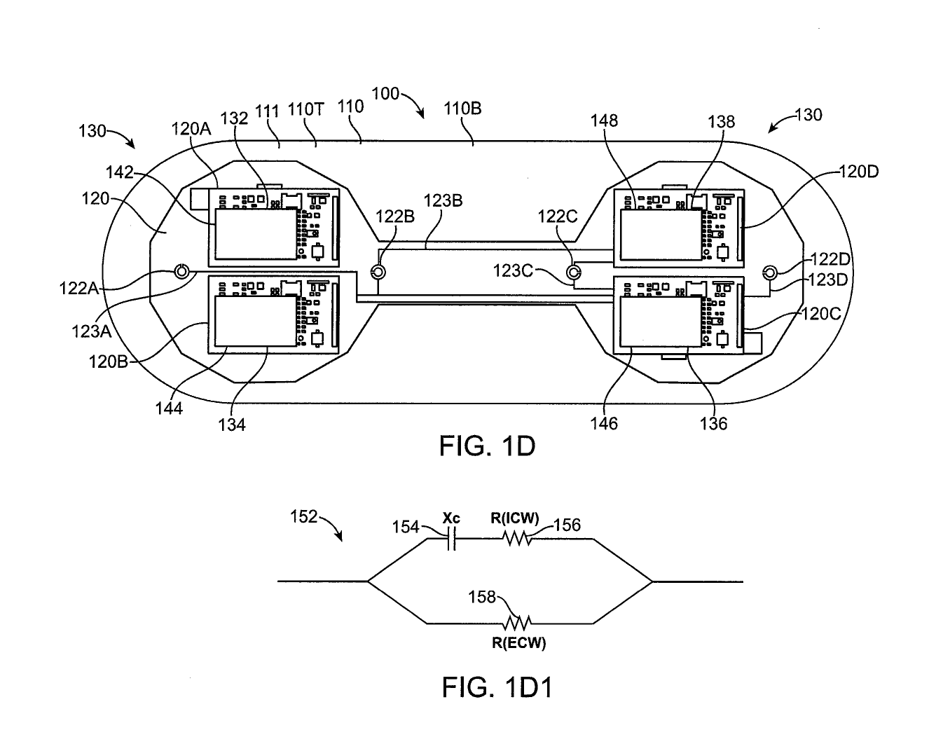

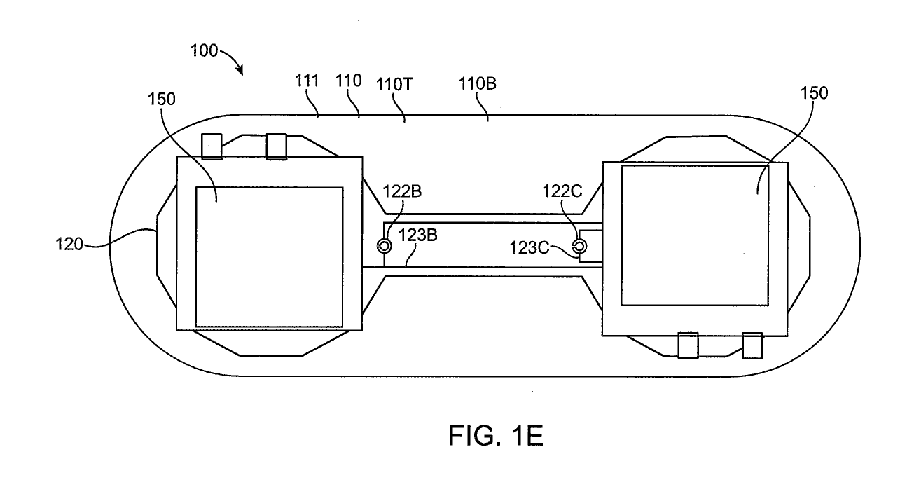

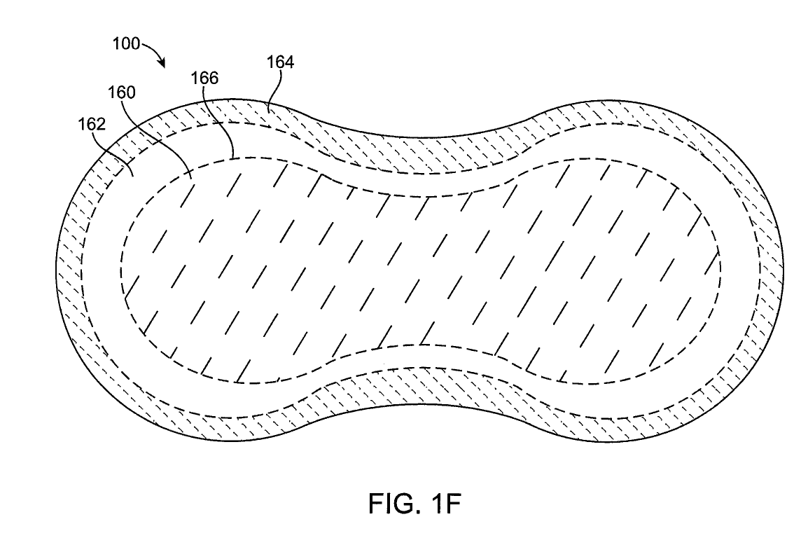

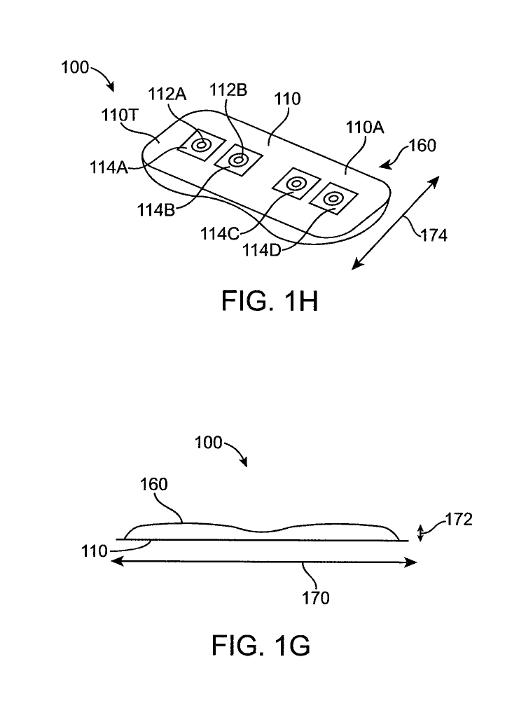

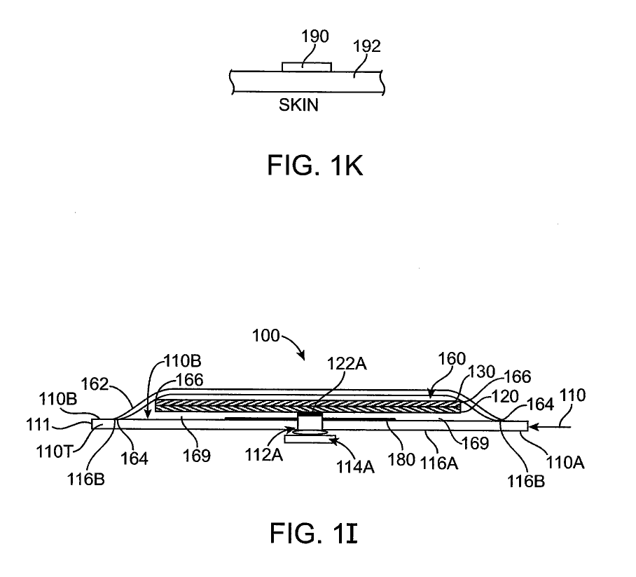

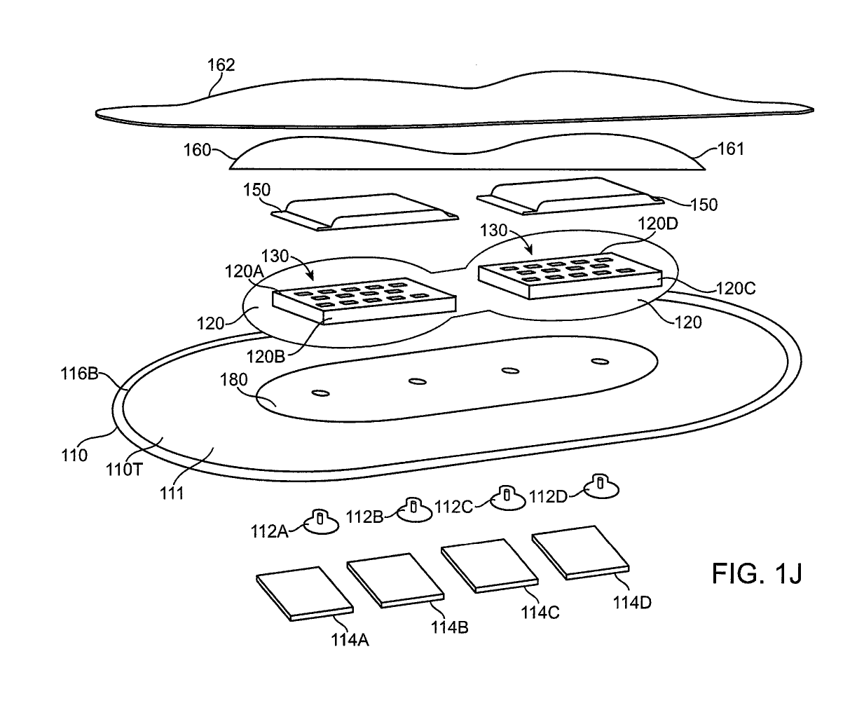

FIG. 1A shows a patient P and a monitoring system 10. Patient P comprises a midline M, a first side S1, for example a right side, and a second side S2, for example a left side. Monitoring system 10 comprises an adherent device 100. Adherent device 100 can be adhered to a patient P at many locations, for example thorax T of patient P. In many embodiments, the adherent device may adhere to one side of the patient, from which side data can be collected. Work in relation with embodiments of the present invention suggests that location on a side of the patient can provide comfort for the patient while the device is adhered to the patient.

Monitoring system 10 includes components to transmit data to a remote center 106. Adherent device 100 can communicate wirelessly to an intermediate device 102, for example with a single wireless hop from the adherent device on the patient to the intermediate device. Intermediate device 102 can communicate with remote center 106 in many ways, for example with an internet connection. In many embodiments, monitoring system 10 comprises a distributed processing system with at least one processor on device 100, at least one processor on intermediate device 102, and at least one process at remote center 106, each of which processors is in electronic communication with the other processors. Remote center 106 can be in communication with a health care provider 108A with a communication system 107A, such as the Internet, an intranet, phone lines, wireless and/or satellite phone. Health care provider 108A, for example a family member, can be in communication with patient P with a communication, for example with a two way communication system, as indicated by arrow 109A, for example by cell phone, email, landline. Remote center 106 can be in communication with a health care professional, for example a physician 108B, with a communication system 107B, such as the Internet, an intranet, phone lines, wireless and/or satellite phone. Physician 108B can be in communication with patient P with a communication, for example with a two way communication system, as indicated by arrow 109B, for example by cell phone, email, landline. Remote center 106 can be in communication with an emergency responder 108C, for example a 911 operator and/or paramedic, with a communication system 107C, such as the Internet, an intranet, phone lines, wireless and/or satellite phone. Emergency responder 108C can travel to the patient as indicated by arrow 109C. Thus, in many embodiments, monitoring system 10 comprises a closed loop system in which patient care can be monitored and implemented from the remote center in response to signals from the adherent device.

In many embodiments, the adherent device may continuously monitor physiological parameters, communicate wirelessly with a remote center, and provide alerts when necessary. The system may comprise an adherent patch, which attaches to the patient's thorax and contains sensing electrodes, battery, memory, logic, and wireless communication capabilities. In some embodiments, the patch can communicates with the remote center, via the intermediate device in the patient's home. In the many embodiments, the remote center receives the data and applies the prediction algorithm. When a flag is raised, the center may communicate with the patient, hospital, nurse, and/or physician to allow for therapeutic intervention to prevent decompensation.

The adherent device may be affixed and/or adhered to the body in many ways. For example, with at least one of the following an adhesive tape, a constant-force spring, suspenders around shoulders, a screw-in microneedle electrode, a pre-shaped electronics module to shape fabric to a thorax, a pinch onto roll of skin, or transcutaneous anchoring. Patch and/or device replacement may occur with a keyed patch (e.g. two-part patch), an outline or anatomical mark, a low-adhesive guide (place guide|remove old patch|place new patch|remove guide), or a keyed attachment for chatter reduction. The patch and/or device may comprise an adhesiveless embodiment (e.g. chest strap), and/or a low-irritation adhesive model for sensitive skin. The adherent patch and/or device can comprise many shapes, for example at least one of a dogbone, an hourglass, an oblong or an oval shape.

In many embodiments, the adherent device may comprise a reusable electronics module with replaceable patches (the module collects cumulative data for approximately 90 days) and/or the entire adherent component (electronics+patch) may be disposable. In a completely disposable embodiment, a “baton” mechanism may be used for data transfer and retention, for example baton transfer may include baseline information. In some embodiments, the device may have a rechargeable module, and may use dual battery and/or electronics modules, wherein one module 101A can be recharged using a charging station 103 while the other module 101B is placed on the adherent device. In some embodiments, the intermediate device 102 may comprise the charging module, data transfer, storage and/or transmission, such that one of the electronics modules can be placed in the intermediate device for charging and/or data transfer while the other electronics module is worn by the patient.

In many embodiments, the system can perform the following functions: initiation, programming, measuring, storing, analyzing, communicating, predicting, and displaying. The adherent device may contain a subset of the following physiological sensors: bioimpedance, respiration, respiration rate variability, heart rate (ave, min, max), heart rhythm, HRV, HRT, heart sounds (e.g. S3), respiratory sounds, blood pressure, activity, posture, wake/sleep, orthopnea, temperature/heat flux, and weight. The activity sensor may be one of the following: ball switch, accelerometer, minute ventilation, HR, bioimpedance noise, skin temperature/heat flux, BP, muscle noise, posture.

In many embodiments, the patch wirelessly communicates with a remote center. In some embodiments, the communication may occur directly (via a cellular or Wi-Fi network), or indirectly through intermediate device 102. Intermediate device 102 may consist of multiple devices which communicate wired or wirelessly to relay data to remote center 106.

In many embodiments, instructions are transmitted from a remote site to a processor supported with the patient, and the processor supported with the patient can receive updated instructions for the patient treatment and/or monitoring, for example while worn by the patient.

FIG. 1B shows a bottom view of adherent device 100 as in FIG. 1A comprising an adherent patch 110. Adherent patch 110 comprises a first side, or a lower side 110A, that is oriented toward the skin of the patient when placed on the patient. In many embodiments, adherent patch 110 comprises a tape 110T which is a material, preferably breathable, with an adhesive 116A. Patient side 110A comprises adhesive 116A to adhere the patch 110 and adherent device 100 to patient P. Electrodes 112A, 112B, 112C and 112D are affixed to adherent patch 110. In many embodiments, at least four electrodes are attached to the patch, for example six electrodes. In some embodiments the patch comprises two electrodes, for example two electrodes to measure an electrocardiogram (ECG) of the patient. Gel 114A, gel 114B, gel 114C and gel 114D can each be positioned over electrodes 112A, 112B, 112C and 112D, respectively, to provide electrical conductivity between the electrodes and the skin of the patient. In many embodiments, the electrodes can be affixed to the patch 110, for example with known methods and structures such as rivets, adhesive, stitches, etc. In many embodiments, patch 110 comprises a breathable material to permit air and/or vapor to flow to and from the surface of the skin.

FIG. 1C shows a top view of the adherent patch 100, as in FIG. 1B. Adherent patch 100 comprises a second side, or upper side 110B. In many embodiments, electrodes 112A, 112B, 112C and 112D extend from lower side 110A through the adherent patch to upper side 110B. In some embodiments, an adhesive 116B can be applied to upper side 110B to adhere structures, for example a cover, to the patch such that the patch can support the electronics and other structures when the patch is adhered to the patient. The PCB comprise completely flex PCB, rigid PCB combined flex PCB and/or rigid PCB boards connected by cable.

FIG. 1D shows a printed circuit boards and electronic components over adherent patch 110, as in FIG. 1C. In some embodiments, a printed circuit board (PCB), PCB 120, for example flex PCB, may be positioned above upper side 100B of patch 110 with connectors 122A, 122B, 122C and 122D. PCB 120 can include traces 123A, 123B, 123C and 123D that extend to connectors 122A, 122B, 122C and 122D, respectively, on the PCB. In some embodiments, the PCB can be rigid with a flex circuit and/or cable connectors. In some embodiments, the PCB may comprise a flex PCB with rigid stiffeners under the electronics components. Connectors 122A, 122B, 122C and 122D can be positioned on PCB 120 in alignment with electrodes 112A, 112B, 112C and 112D so as to electrically couple the PCB with the electrodes. In some embodiments, connectors 122A, 122B, 122C and 122D may comprise insulated wires that provide strain relief between the PCB and the electrodes. In some embodiments, additional PCB's, for example rigid PCB's 120A, 120B, 120C and 120D can be connected to PCB 120. Electronic components 130 can be connected to PCB 120 and/or mounted thereon. In some embodiments, electronic components 130 can be mounted on the additional PCB's.

Electronic components 130 comprise components to take physiologic measurements, transmit data to remote center 106 and receive commands from remote center 106. In many embodiments, electronics components 130 may comprise known low power circuitry, for example complementary metal oxide semiconductor (CMOS) circuitry components. Electronics components 130 comprise an activity sensor and activity circuitry 134, impedance circuitry 136 and electrocardiogram circuitry, for example ECG circuitry 138. In some embodiments, electronics circuitry 130 may comprise a microphone and microphone circuitry 142 to detect an audio signal from within the patient, and the audio signal may comprise a heart sound and/or a respiratory sound, for example an S3 heart sound and a respiratory sound with rales and/or crackles. Electronics circuitry 130 may comprise a temperature sensor, for example a thermistor, and temperature sensor circuitry 144 to measure a temperature of the patient, for example a temperature of a skin of the patient.

Work in relation to embodiments of the present invention suggests that skin temperature may effect impedance and/or hydration measurements, and that skin temperature measurements may be used to correct impedance and/or hydration measurements. In some embodiments, increase in skin temperature can be associated with increased vaso-dilation near the skin surface, such that measured impedance measurement decreased, even through the hydration of the patient in deeper tissues under the skin remains substantially unchanged. Thus, use of the temperature sensor can allow for correction of the hydration signals to more accurately assess the hydration, for example extra cellular hydration, of deeper tissues of the patient, for example deeper tissues in the thorax.

Work in relation to embodiments of the present invention suggests that patient body position and/or activity may effect impedance and/or hydration measurements, and that accelerometer signals may be used to correct impedance and/or hydration measurements. In some embodiments, increase in patient activity can be associated with increased vaso-dilation near the skin surface, similar to temperature measurements. Thus, use of the accelerometer signals and/or temperature sensor signals can allow for correction of the hydration signals to more accurately assess the hydration, for example extra cellular hydration, of deeper tissues of the patient, for example deeper tissues in the thorax.

Electronics circuitry 130 may comprise a processor 146. Processor 146 comprises a tangible medium, for example read only memory (ROM), electrically erasable programmable read only memory (EEPROM) and/or random access memory (RAM). Electronic circuitry 130 may comprise real time clock and frequency generator circuitry 148. In some embodiments, processor 136 may comprise the frequency generator and real time clock. The processor can be configured to control a collection and transmission of data from the impedance circuitry electrocardiogram circuitry and the accelerometer. In many embodiments, device 100 comprise a distributed processor system, for example with multiple processors on device 100.

In many embodiments, electronics components 130 comprise wireless communications circuitry 132 to communicate with remote center 106. The wireless communication circuitry can be coupled to the impedance circuitry, the electrocardiogram circuitry and the accelerometer to transmit to a remote center with a communication protocol at least one of the hydration signal, the electrocardiogram signal or the accelerometer signal. In specific embodiments, wireless communication circuitry is configured to transmit the hydration signal, the electrocardiogram signal and the accelerometer signal to the remote center with a single wireless hop, for example from wireless communication circuitry 132 to intermediate device 102. The communication protocol comprises at least one of Bluetooth, Zigbee, WiFi, WiMax, IR, amplitude modulation or frequency modulation. In many embodiments, the communications protocol comprises a two way protocol such that the remote center is capable of issuing commands to control data collection.

In some embodiments, intermediate device 102 comprises a data collection system to collect and store data from the wireless transmitter. The data collection system can be configured to communicate periodically with the remote center. In many embodiments, the data collection system can transmit data in response to commands from remote center 106 and/or in response to commands from the adherent device.

Activity sensor and activity circuitry 134 can comprise many known activity sensors and circuitry. In many embodiments, the accelerometer comprises at least one of a piezoelectric accelerometer, capacitive accelerometer or electromechanical accelerometer. The accelerometer may comprises a 3-axis accelerometer to measure at least one of an inclination, a position, an orientation or acceleration of the patient in three dimensions. Work in relation to embodiments of the present invention suggests that three dimensional orientation of the patient and associated positions, for example sitting, standing, lying down, can be very useful when combined with data from other sensors, for example ECG data and/or hydration data.

Impedance circuitry 136 can generate both hydration data and respiration data. In many embodiments, impedance circuitry 136 is electrically connected to electrodes 112A, 112B, 112C and 112D such that electrodes 112A and 112D comprise outer electrodes that are driven with a current, or force electrodes. The current delivered between electrodes 112A and 112D generates a measurable voltage between electrodes 112B and 112C, such that electrodes 112B and 112C comprise inner electrodes, or sense electrodes that measure the voltage in response to the current from the force electrodes. The voltage measured by the sense electrodes can be used to determine the hydration of the patient.

FIG. 1D1 shows an equivalent circuit 152 that can be used to determine optimal frequencies for measuring patient hydration. Work in relation to embodiments of the present invention indicates that the frequency of the current and/or voltage at the force electrodes can be selected so as to provide impedance signals related to the extracellular and/or intracellular hydration of the patient tissue. Equivalent circuit 152 comprises an intracellular resistance 156, or R(ICW) in series with a capacitor 154, and an extracellular resistance 158, or R(ECW). Extracellular resistance 158 is in parallel with intracellular resistance 156 and capacitor 154 related to capacitance of cell membranes. In many embodiments, impedances can be measured and provide useful information over a wide range of frequencies, for example from about 0.5 kHz to about 200 KHz. Work in relation to embodiments of the present invention suggests that extracellular resistance 158 can be significantly related extracellular fluid and to cardiac decompensation, and that extracellular resistance 158 and extracellular fluid can be effectively measured with frequencies in a range from about 0.5 kHz to about 20 kHz, for example from about 1 kHz to about 10 kHz. In some embodiments, a single frequency can be used to determine the extracellular resistance and/or fluid. As sample frequencies increase from about 10 kHz to about 20 kHz, capacitance related to cell membranes decrease the impedance, such that the intracellular fluid contributes to the impedance and/or hydration measurements. Thus, many embodiments of the present invention employ measure hydration with frequencies from about 0.5 kHz to about 20 kHz to determine patient hydration.

In many embodiments, impedance circuitry 136 can be configured to determine respiration of the patient. In specific embodiments, the impedance circuitry can measure the hydration at 25 Hz intervals, for example at 25 Hz intervals using impedance measurements with a frequency from about 0.5 kHz to about 20 kHz.

ECG circuitry 138 can generate electrocardiogram signals and data from electrodes 112A, 112B, 112C and 112D. In some embodiments, ECG circuitry 138 is connected to inner electrodes 112B and 122C, which may comprise sense electrodes of the impedance circuitry as described above. In some embodiments, the inner electrodes may be positioned near the outer electrodes to increase the voltage of the ECG signal measured by ECG circuitry 138. In some embodiments, the ECG circuitry can share components with the impedance circuitry.

FIG. 1E shows batteries 150 positioned over the flex printed circuit board and electronic components as in FIG. 1D. Batteries 150 may comprise rechargeable batteries that can be removed and/or recharged. In some embodiments, batteries 150 can be removed from the adherent patch and recharged and/or replaced.

FIG. 1F shows a top view of a cover 162 over the batteries, electronic components and flex printed circuit board as in FIG. 1E. In many embodiments, an electronics housing 160 may be disposed under cover 162 to protect the electronic components, and in some embodiments electronics housing 160 may comprise an encapsulant, for example a dip coating, over the electronic components and PCB. In some embodiments, cover 162 can be adhered to adhesive patch with an adhesive 164 on an underside of cover 162. In some embodiments, electronics housing 160 can be adhered to cover 162 with an adhesive 166 where cover 162 contacts electronics housing 160. In many embodiments, electronics housing 160 may comprise a water proof material, for example a sealant adhesive such as epoxy or silicone coated over the electronics components and/or PCB. In some embodiments, electronics housing 160 may comprise metal and/or plastic.

Cover 162 may comprise many known biocompatible cover, casing and/or housing materials, such as elastomers, for example silicone. The elastomer may be fenestrated to improve breathability. In some embodiments, cover 162 may comprise many known breathable materials, for example polyester and/or polyamide fabric with 5 to 25% elastane/spandex. The breathable fabric may be coated to make it water resistant, waterproof, and/or to aid in wicking moisture away from the patch.

FIG. 1G shows a side view of adherent device 100 as in FIGS. 1A to 1F. Adherent device 100 comprises a maximum dimension, for example a length 170 from about 4 to 10 inches (from about 100 mm to about 250 mm), for example from about 6 to 8 inches (from about 150 mm to about 200 mm). In some embodiments, length 170 may be no more than about 6 inches (no more than about 150 mm). Adherent device 100 comprises a thickness 172. Thickness 172 may comprise a maximum thickness along a profile of the device. Thickness 172 can be from about 0.2 inches to about 0.4 inches (from about 5 mm to about 10 mm), for example about 0.3 inches (about 7.5 mm).

FIG. 1H shows a bottom isometric view of adherent device 100 as in FIGS. 1A to 1G. Adherent device 100 comprises a width 174, for example a maximum width along a width profile of adherent device 100. Width 174 can be from about 2 to about 4 inches (from about 50 mm to 100 mm), for example about 3 inches (about 75 mm).

FIGS. 1I and 1J show a side cross-sectional view and an exploded view, respectively, of adherent device 100 as in FIGS. 1A to 1H. Device 100 comprises several layers. Gel 114A, or gel layer, is positioned on electrode 112A to provide electrical conductivity between the electrode and the skin. Electrode 112A may comprise an electrode layer. Adhesive patch 110 may comprise a layer of breathable tape 110T, for example a known breathable tape, such as tricot-knit polyester fabric. An adhesive 116A, for example a layer of acrylate pressure sensitive adhesive, can be disposed on underside 110A of patch 110. A gel cover 180, or gel cover layer, for example a polyurethane non-woven tape, can be positioned over patch 110 comprising the breathable tape. PCB 120, for example a flex PCB, or flex PCB layer, can be positioned over gel cover 180 with electronic components 130 connected and/or mounted to PCB 120, for example mounted on flex PCB so as to comprise an electronics layer disposed on the flex PCB. In many embodiments, the adherent device may comprise a segmented inner component, for example the PCB, for limited flexibility. In many embodiments, the electronics layer may be encapsulated in electronics housing 160 which may comprise a waterproof material, for example silicone or epoxy. In many embodiments, the electrodes are connected to the PCB with a flex connection, for example trace 123A of PCB 120, so as to provide strain relive between the electrodes 112A, 112B, 112C and 112D and the PCB. Gel cover 180 can inhibit flow of gel 114A and liquid. In many embodiments, gel cover 180 can inhibit gel 114A from seeping through breathable tape 110T to maintain gel integrity over time. Gel cover 180 can also keep excessive external moisture from penetrating into gel 114A. In many embodiments, cover 162 can encase the flex PCB and/or electronics housing and can be adhered to at least one of the electronics, the PCB or the adherent patch, so as to protect the device. In some embodiments, cover 162 attaches to adhesive patch 110 with adhesive 116B, and cover 162 is adhered to the PCB module with an adhesive 161 on the upper surface of the electronics housing. Cover 162 can comprise many known biocompatible cover, housing and/or casing materials, for example silicone. In many embodiments, cover 162 comprises an outer polymer cover to provide smooth contour without limiting flexibility. In some embodiments, cover 162 may comprise a breathable fabric. Cover 162 may comprise many known breathable fabrics, for example breathable fabrics as described above. In some embodiments, the breathable fabric may comprise polyester, polyamide, and/or elastane (Spandex) to allow the breathable fabric to stretch with body movement. In some embodiments, the breathable tape may contain and elute a pharmaceutical agent, such as an antibiotic, anti-inflammatory or antifungal agent, when the adherent device is placed on the patient.

In many embodiments, the breathable tape of adhesive patch 110 comprises a first mesh with a first porosity and gel cover 180 comprises a breathable tape with a second mesh porosity, in which the second porosity is less than the first porosity to inhibit flow of the gel through the breathable tape.

In many embodiments, a gap 169 extends from adherent patch 110 to the electronics module and/or PCB, such that breathable tape 110T can breath when the patch is applied to the patient so as to provide patient comfort.

In many embodiments, the adherent device comprises a patch component and at least one electronics module. The patch component may comprise adhesive patch 110 comprising the breathable tape with adhesive coating 116A, at least one electrode 112A and gel 114A, for example a gel coating. The at least one electronics module can be separable from the patch component. In many embodiments, the at least one electronics module comprises the printed circuit board 120, electronic component 130, and electronics housing 160, such that the printed circuit board, electronic components, electronics housing and water proof cover are reusable and/or removable for recharging and data transfer, for example as described above. In many embodiments, adhesive 116B is coated on upper side 110A of adhesive patch 110B, such that the cover can be adhered to the patch. In specific embodiments, the electronic module can be attached to the patch component with a releasable connection, for example with Velcro™, a known hook and loop connection, and/or snap directly to the electrodes. In some embodiments, two electronics modules can be provided, such that one electronics module can be worn by the patient while the other is charged as described above.

In many embodiments, at least one electrode 112A extends through at least one aperture in the breathable tape 110T.

In some embodiments, the adherent patch, for example an adhesive patch, may comprise a medicated patch that releases a medication, such as antibiotic, beta-blocker, ACE inhibitor, diuretic, or steroid to reduce skin irritation. In some embodiments, the adhesive patch may comprise a thin, flexible, breathable patch with a polymer grid for stiffening. This grid may be anisotropic, may use electronic components to act as a stiffener, may use electronics-enhanced adhesive elution, and may use an alternating elution of adhesive and steroid.

FIG. 1K shows at least one electrode 190 configured to electrically couple to a skin of the patient through a breathable tape 192. In many embodiments, at least one electrode 190 and breathable tape 192 comprise electrodes and materials similar to those described above. Electrode 190 and breathable tape 192 can be incorporated into adherent devices as described above, so as to provide electrical coupling between the skin and electrode through the breathable tape, for example with the gel.

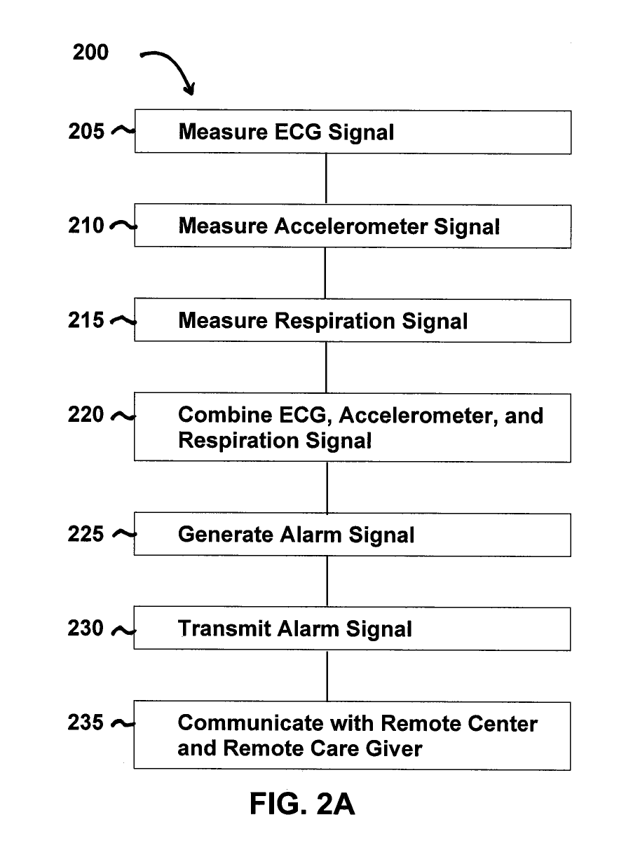

FIG. 2A shows a method 200 of monitoring a patient. A step 205 measures an electrocardiogram signal. A step 210 measures an accelerometer signal and a temperature signal. A step 215 measures a respiration signal. A step 220 combines at least two of the electrocardiogram signal, the accelerometer signal, the temperature signal and respiration signal. A step 225 generates an alarm signal. In many embodiments, the alarm signal may be generated in response to a detected patient fall and/or decreased patient activity from the accelerometer signal; an increased heart rate measured with the electrocardiogram signal and/or abnormal respiratory rate; and/or respiratory distress from the respiration signal. A step 230 transmits the alarm signal. In many embodiments, a step may also comprise of transmitting at least one of the electrocardiogram signal, accelerometer, and respiration signal. In some embodiments, transmission may occur in real time. In some embodiments, transmissions may be performed by wireless communication circuitry with a single wireless hop from the wireless communication circuitry to an intermediate device and a wireless hop from the intermediate device to the remote center. A step 235 communicates with a remote center and/or remote care giver.

As mentioned above, a step 220 combines at least two of the electrocardiogram, accelerometer, and respiration signal. The signals can be combined in many ways. In some embodiments, the signals can be combined by using the at least two of the electrocardiogram, accelerometer, and respiration signal to look up a value in a previously existing array.

TABLE 1
Lookup Table for ECG and Hydration Signals

| | | | |
|---|---|---|---|
| Heart Rate/Hydration | 0-49 bpm | 50-69 bpm | 70-90 bpm |

| | | | | |
|---|---|---|---|---|
| >60 | Ohms | N | N | Y |
| 41-59 | Ohms | N | Y | Y |
| 0-40 | Ohms | Y | Y | Y |

The look up table shown in Table 1 illustrates the use of a look up table according to one embodiment, and one will recognize that many variables can be combined with a look up table. For example at a heart rate of 89 bpm and a hydration of 35 Ohms, the value in the table may comprise Y. In specific embodiments, the values of the look up table can be determined in response to empirical data measured for a patient population, for example measurements on about 1000 to 10,000 patients.

In some embodiments, the table may comprise a three or more dimensional look up table, and the look up table may comprise a tier, or level, of the response, for example an alarm.

In some embodiments, the signals may be combined with at least one of adding, subtracting, multiplying, scaling or dividing the at least two of the electrocardiogram signal, the respiration signal or the activity signal. In specific embodiments, the measurement signals can be combined with positive and or negative coefficients determined in response to empirical data measured for a patient population, for example data on about 1000 to 10,000 patients.

In some embodiments, a weighted combination may combine at least 3 measurement signals to generate an output value according to a formula of the general form

OUTPUT=aX+bY

where a and b comprise positive or negative coefficients determined from empirical data and X, and Y comprise measured signals for the patient, for example at least two of the electrocardiogram, accelerometer, and respiration signal. While two coefficients and two variables are shown, the data may be combined with multiplication and/or division. One or more of the variables may be the inverse of a measured variable.

In some embodiments, the data may be combined with a tiered combination. While many tiered combinations can be used a tiered combination with three measurement signals can be expressed as

In some embodiments, the ECG signal comprises a heart rate signal that can be divided by the accelerometer signal. Work in relation to embodiments of the present invention suggest that an increase in heart rate with a decrease in activity can indicate an impending decompensation. The signals can be combined to generate an output value with an equation of the general form

OUTPUT=aX/Y+bZ

where X comprise a heart rate signal, Y comprises a accelerometer rate signal and Z comprises a respiration signal, with each of the coefficients determined in response to empirical data as described above.

OUTPUT=(ΔX)+(ΔY)+(ΔZ)

where (ΔX), (ΔY), (ΔZ) may comprise change in heart rate signal from baseline, change in accelerometer signal from baseline and change in respiration signal from baseline, and each may have a value of zero or one, based on the values of the signals. For example if the heart rate increase by 10%, (ΔX) can be assigned a value of 1. If the accelerometer signal increases by 5%, (ΔY) can be assigned a value of 1. If the respiration signal decreases below 10% of a baseline value (ΔZ) can be assigned a value of 1. When the output signal is three, a flag may be set to trigger an alarm.

In some embodiments, the data may be combined with a logic gated combination. While many logic gated combinations can be used a logic gated combination with three measurement signals can be expressed as

OUTPUT=(ΔX) AND (ΔY) AND (ΔZ)

where (ΔX), (ΔY), (ΔZ) may comprise change in heart rate signal from baseline, change in accelerometer signal from baseline and change in respiration signal from baseline, and each may have a value of zero or one, based on the values of the signals. For example if the heart rate increase by 10%, (ΔX) can be assigned a value of 1. If the accelerometer signal increases by 5%, (ΔY) can be assigned a value of 1. If the respiration signal decreases below 10% of a baseline value (ΔZ) can be assigned a value of 1. When each of (ΔX), (ΔY), (ΔZ) is one, the output signal is one, and a flag may be set to trigger an alarm. If any one of (ΔX), (ΔY) or (ΔZ) is zero, the output signal is zero and a flag may be set so as not to trigger an alarm. While a specific example with AND gates has been shown the data can be combined in may ways with known gates for example NAND, NOR, OR, NOT, XOR, XNOR gates. In some embodiments, the gated logic may be embodied in a truth table.

It should be appreciated that the specific steps performed as described above and illustrated in FIG. 2A provide a particular method of monitoring a patient, according to an embodiment of the present invention. Other sequences of steps may also be performed according to alternative embodiments. For example, alternative embodiments of the present invention may perform the steps outlined above in a different order. Moreover, the individual steps illustrated in FIG. 2A may include multiple sub-steps that may be performed in various sequences as appropriate to the individual step. Furthermore, additional steps may be added or removed depending on the particular applications. One of ordinary skill in the art would recognize many variations, modifications, and alternatives.

While the exemplary embodiments have been described in some detail, by way of example and for clarity of understanding, those of skill in the art will recognize that a variety of modifications, adaptations, and changes may be employed. Hence, the scope of the present invention should be limited solely by the appended claims.

What is claimed is:

1. An adherent device to monitor a person, the device comprising:
an adhesive patch to adhere to a skin of the person;
at least two electrodes connected to the patch and capable of electrically coupling to the person;
electrocardiogram circuitry coupled to at the least two electrodes to measure an electrocardiogram signal of the person;
an accelerometer mechanically coupled to the adhesive patch to generate an accelerometer signal in response to at least one of an activity or a position of the person; and
a processor comprising a tangible medium and configured to communicate with the electrocardiogram circuitry and the accelerometer to generate an alarm signal in response to the electrocardiogram signal and the accelerometer signal.

2. The adherent device of claim 1 wherein the processor is configured to transmit at least one of the electrocardiogram signal or the accelerometer signal in real time to the remote center and/or a remote care giver in response to the alarm.

3. The adherent device of claim 1 wherein the processor is configured to generate the alarm signal in response to at least one of a cardiac rhythm disorder, a fall, a temperature or a respiratory distress of the person.

4. The adherent device of claim 1 wherein the processor is configured to generate the alarm signal in response to a detected person fall from the accelerometer signal

5. The adherent device of claim 1 wherein the processor is configured to generate the alarm signal in response to an increased heart rate from the electrocardiogram signal and a decreased person activity from the accelerometer signal.

6. The adherent device of claim 1 further comprising respiration circuitry to measure a respiration signal of the person and wherein the processor is configured to generate the alarm signal in response to a respiratory distress from the respiration signal.

7. The adherent device of claim 6 wherein the processor is configured to combine the electrocardiogram signal, the accelerometer signal and respiration signal to generate the alarm signal.

8. The adherent device of claim 7 wherein combining comprises using the at least two of the electrocardiogram signal, the accelerometer signal, or the respiration signal to look up a value in a previously existing array.

9. The adherent device of claim 7 wherein combining comprises at least one of adding, subtracting, multiplying, scaling or dividing the at least two of the electrocardiogram signal, the accelerometer signal, or the respiration signal.

10. The adherent device of claim 7 wherein the at least two of the electrocardiogram signal, the accelerometer signal, or the respiration signal are combined with at least one of a weighted combination, a tiered combination or a logic gated combination, a time weighted combination or a rate of change.

11. The adherent device of claim 6 wherein the processor is configured to generate the alarm signal in response to an abnormal respiratory rate from the respiration signal and a decreased person activity from the accelerometer signal.

12. The adherent device of claim 6 wherein the processor is configured to generate the alarm signal in response to an abnormal heart rate from the electrocardiogram signal, an abnormal respiratory rate from the respiration signal, and a decreased activity measured from the accelerometer signal.

13. The adherent device of claim 6 wherein the respiration circuitry comprises at least one of an impedance circuitry or a strain gauge.

14. The adherent device of claim 6 wherein the adhesive patch is mechanically coupled to the at two electrodes, the electrocardiogram circuitry, the accelerometer, the respiration circuitry and the processor, such that the patch is capable of supporting the at least two electrodes, the electrocardiogram circuitry, the respiration circuitry, the accelerometer and the processor when the adherent patch is adhered to the skin of the person.

15. The adherent device of claim 1 further comprising a wireless communication circuitry coupled to the processor transmit at least one of the electrocardiogram signal, the respiration signal, or the accelerometer signal to a remote center with a communication protocol.

16. The adherent device of claim 15 wherein wireless communication circuitry is configured to transmit the at least one of the electrocardiogram signal, the respiration signal or the accelerometer signal to the remote center with a single wireless hop from the wireless communication circuitry to an intermediate device and a wireless hop from the intermediate device to the remote center.

17. A method of monitoring a frail person, the method comprising:
adhering an adhesive patch to a skin of the person, such that at least two electrodes connected to the patch are coupled to the skin of the person;
measuring an electrocardiogram signal of the person with electrocardiogram circuitry coupled to at the least two electrodes;
measuring an accelerometer signal in response to at least one of an activity or a position of the person with an accelerometer mechanically coupled to the adhesive patch; and
generating an alarm signal in response to the electrocardiogram signal and the accelerometer signal with a processor comprising a tangible medium and in communication with the electrocardiogram circuitry and the accelerometer.

18. The method of claim 17 further comprising transmitting at least one of the electrocardiogram signal or the accelerometer signal in real time to the remote center and/or a remote care giver in response to the alarm.

19. The method of claim 17 wherein the alarm signal is generated in response to at least one of a cardiac rhythm disorder, a fall or a respiratory distress of the person.

20. The method of claim 17 wherein the alarm signal is generated in response to a detected person fall from the accelerometer signal

21. The method of claim 17 wherein the alarm signal is generated in response to an increased heart rate measured with the electrocardiogram signal and a decreased person activity measured with the accelerometer signal.

22. The method of claim 17 wherein the alarm signal is generated in response to a decreased heart rate measured with the electrocardiogram signal and a decreased person activity measured with the accelerometer signal so as to indicate at least one of a syncope and/or fainting of the person.

23. The method of claim 17 further comprising measuring a respiration signal of the person with respiration circuitry and wherein the alarm signal is generated in response to a respiratory distress from the respiration signal.

24. The method of claim 23 wherein the electrocardiogram signal, the accelerometer signal and respiration signal are combined to generate the alarm signal.

25. The method of claim 24 wherein combining comprises using the at least two of the electrocardiogram signal, the accelerometer signal, or the respiration signal to look up a value in a previously existing array.

26. The method of claim 24 wherein combining comprises at least one of adding, subtracting, multiplying, scaling or dividing the at least two of the electrocardiogram signal, the accelerometer signal, or the respiration signal.

27. The method of claim 24 wherein the at least two of the electrocardiogram signal, the accelerometer signal, or the respiration signal are combined with at least one of a weighted combination, a tiered combination or a logic gated combination, a time weighted combination or a rate of change.

28. The method of claim 23 wherein the alarm signal is generated in response to an abnormal respiratory rate from the respiration signal and a decreased person activity from the accelerometer signal.

29. The method of claim 23 wherein the alarm signal is generated in response to an abnormal heart rate from the electrocardiogram signal, an abnormal respiratory rate from the respiration signal, and a decreased activity from the accelerometer signal.

30. The method of claim 23 wherein the adhesive patch is mechanically coupled to the at two electrodes, the electrocardiogram circuitry, the accelerometer, the respiration circuitry and the processor, such that the patch supports the at least two electrodes, the electrocardiogram circuitry, the respiration circuitry, the accelerometer and the processor when the adherent patch is adhered to the skin of the person.

31. The method of claim 17 further comprising transmitting wirelessly at least one of the electrocardiogram signal, the respiration signal, or the accelerometer signal to a remote center with a communication protocol.

32. The method of claim 31 wherein the at least one of the electrocardiogram signal, the respiration signal or the accelerometer signal is transmitted to the remote center with a single wireless hop from the wireless communication circuitry to an intermediate device and a wireless hop from the intermediate device to the remote center.

33. An adherent device to monitor a person in an emergency situation, the device comprising:
an adhesive patch to adhere to a skin of the person;
at least two electrodes connected to the patch and capable of electrically coupling to the person;
electrocardiogram circuitry coupled to at the least two electrodes to measure an electrocardiogram signal of the person;
temperature circuitry to measure a temperature signal from the patient;
respiration circuitry to measure a respiration signal of the person;
a processor comprising a tangible medium and configured to communicate with the electrocardiogram circuitry, the temperature circuitry and the respiration circuitry to generate an alarm signal in response to at least two of the electrocardiogram signal, the temperature signal or the respiration signal; and
wireless communication circuitry coupled to the processor and the electrocardiogram circuitry and the accelerometer to transmit the alarm signal to a remote center with a communication protocol.

34. The adherent device of claim 33 further comprising temperature circuitry to measure a temperature signal from the patient.

35. The adherent device of claim 33 wherein the processor is configured to transmit at least one of the electrocardiogram signal or the respiration signal in real time to the remote center and/or a remote care giver in response to the alarm.

36. The adherent device of claim 33 wherein the wireless communication circuitry is configured to transmit at least one of the electrocardiogram signal or the respiration signal in real time to the remote center in response to the alarm signal.

37. The adherent device of claim 33 wherein the respiration circuitry comprises at least one of an impedance circuitry or a strain gauge.

38. The adherent device of claim 33 wherein the processor is configured to generate the alarm signal in response to an abnormal heart rate from the electrocardiogram signal and an abnormal respiratory rate from the respiration signal.

39. The adherent device of claim 33 further comprising an accelerometer mechanically coupled to the adhesive patch to generate an accelerometer signal in response to at least one of an activity or a position of the person.

40. The adherent device of claim 39 wherein the processor is configured to combine the electrocardiogram signal, the accelerometer signal and respiration signal to generate the alarm signal.

41. The adherent device of claim 40 wherein the processor is configured to combine the at least two of the e electrocardiogram signal, the accelerometer signal, the temperature signal or the respiration signal to look up a value in a previously existing array.

42. The adherent device of claim 40 wherein the processor is configured to combine with at least one of adding, subtracting, multiplying, scaling or dividing the at least two of the electrocardiogram signal, the accelerometer signal, the temperature signal or the respiration signal.

43. The adherent device of claim 40 wherein the at least two of the electrocardiogram signal, the accelerometer signal, the temperature signal or the respiration signal are combined with at least one of a weighted combination, a tiered combination or a logic gated combination, a time weighted combination or a rate of change.

44. The adherent device of claim 39 wherein the adhesive patch is mechanically coupled to the at two electrodes, the electrocardiogram circuitry, the respiration circuitry, the accelerometer, the temperature circuitry and the processor, such that the patch is capable of supporting the at least two electrodes, the electrocardiogram circuitry, the respiration circuitry, the accelerometer, the temperature circuitry and the processor when the adherent patch is adhered to the skin of the person.

45. A method of monitoring a person in an emergency situation, the method comprising:
adhering an adhesive patch to a skin of the person, such that at least two electrodes connected to the patch are coupled to the skin of the person;
measuring an electrocardiogram signal of the person with electrocardiogram circuitry coupled to at the least two electrodes;
measuring a respiration signal of the person with respiration circuitry;
measuring a temperature signal from the person with temperature circuitry;
generating an alarm signal in response to the electrocardiogram signal and the respiration signal with a processor comprising a tangible medium in communication with the electrocardiogram circuitry and the respiration circuitry; and
transmitting the alarm signal to a remote center with a communication protocol and wireless communication circuitry.

46. The method of claim 49 wherein at least one of the electrocardiogram signal or the respiration signal are transmitted in real time to the remote center and/or a remote care giver in response to the alarm.

47. The method of claim 49 wherein the alarm signal is generated with the processor in response to an abnormal heart rate from the electrocardiogram signal and an abnormal respiratory rate from the respiration signal.

48. The method of claim 49 further comprising generating an accelerometer signal in response to at least one of an activity or a position of the person with an accelerometer mechanically coupled to the adhesive patch.

49. The method of claim 49 wherein further comprising combing the electrocardiogram signal, the accelerometer signal, the temperature signal and respiration signal to generate the alarm signal with the processor.

50. The method of claim 49 wherein the adhesive patch is mechanically coupled to the at two electrodes, the electrocardiogram circuitry, the respiration circuitry, the accelerometer and the processor, such that the patch supports the at least two electrodes, the electrocardiogram circuitry, the respiration circuitry, the accelerometer and the processor when the adherent patch is adhered to the skin of the person.
